(19) United States
(12) Patent Application Publication
(10) Pub. No.: US 2022/0241154 A1
(43) Pub. Date: Aug. 4, 2022

(54) SYSTEM AND METHOD FOR DUAL-FUNCTION SYRINGE DESIGN COMPOUNDING DEVICE FOR PERSONALIZED TREATMENTS

(71) Applicant: AQUAVIT PHARMACEUTICALS, INC., New York, NY (US)

(72) Inventors: Sobin Chang, New York, NY (US); Kwonsooo Chun, New York, NY (US)

(21) Appl. No.: 17/629,294

(22) PCT Filed: Jul. 22, 2020
(86) PCT No.: PCT/US2020/043152

§ 371 Date: Jan. 21, 2022

Related U.S. Application Data
(60) Provisional application No. 62/876,901, filed on Jul. 22, 2019.

(51) Int. Cl. A61J 1/20; A61J 3/00; A61J 1/16; A61M 5/32; A61M 37/00; A61M 5/24; A61M 5/20

(52) U.S. Cl. CPC A61J 1/2096; A61J 3/002; A61J 1/16; A61M 5/20; A61M 37/0015; A61M 5/24; A61M 5/3297

(57) ABSTRACT

The present invention provides a system for preparing active agents for personalized treatment of a subject, wherein the system comprises a housing enclosing an interior space; an inventory structure comprising a plurality of chambers, wherein each chamber is capable of holding a dual-function syringe comprising a predetermined agent to be administered; means for selecting one or more dual-function syringes according to the predetermined active agent; means for moving the selected dual-function syringe to a desired location for transferring of the predetermined agent from the dual-function syringe to a product container or carrier; and means for sequentially transferring a controlled quantity of the predetermined agent from each selected dual-function syringe to the product container or carrier under positive or negative pressure.

### CROSS-REFERENCE TO RELATED APPLICATIONS

This application claims the benefit of U.S. Provisional Appl. No. 62/876,901, filed Jul. 22, 2019, the contents of which are hereby incorporated by reference in their entirety.

### FIELD OF THE INVENTION

The field of the invention relates generally to the fields of pharmaceuticals, personalized medicine, medical aesthetics, skin healthcare, dermatology, bioinstrumentation, bioengineering and biotechnology.

### BACKGROUND OF THE INVENTION

Bioactive compounds have been used for centuries to cure everything from dry skin and the common cold to HIV/AIDS and melanoma. Scientists have pinpointed specific combinations of bioactive compounds that counter particular symptoms, and these combinations must be carefully crafted in order to result in the desired treatment. Furthermore, disease prevention and treatment should be tailored for the individual's needs whenever possible, and each patient's treatment should take into account their specific health concerns.

Various systems are known for compounding various pharmaceutical admixtures in a single container. See, e.g., U.S. Pat. Nos. 7,610,115, 7,194,336, 7,171,992, 6,975,924, 6,951,228, 5,697,407, 5,431,202, 5,085,256, 5,040,699, 4,922,975, 4,789,014, 4,653,010, 4,513,796, and 4,467,844 for various fluid transfer and compounding systems.

There is a need for a new systems and devices which can compound bioactive agents for personalized treatments.

### SUMMARY OF THE INVENTION

It is to be understood that both the foregoing general description of the embodiments and the following detailed description are exemplary, and thus do not restrict the scope of the embodiments.

There is a great benefit to being able to mix precise volumes of different bioactive agents to create therapeutics which may be administered intravenously (IV), intramuscularly (IM), subcutaneously (SC), topically, intradermally, orally, intranasally, through inhalation or other methods of administration. Being able to combine, for example, vitamins, growth hormones, glucosamine, omega-3 fatty acids, insulin and other bioactive agents into personalized treatments for specific patients will simplify and streamline the medication administration process. Or being able to compound Tylenol, OxyContin, and lidocaine for patients who recently got their tonsils removed so that they do not need to take each separately.

Furthermore, being able to combine either solids or liquids to create solutions or capsules that can be administered in innumerable ways leads to a great variety of treatment combinations that can be created. Finally, since the medication requirements vary from patient to patient, it is necessary to develop a personalized formulation of bioactive agents to meet the needs of the patient.

The present invention solves these and other problems by providing systems and methods comprising dual-function syringes comprising bioactive agents. The dual-function syringes can either administer liquid, solid, or gel like bioactive agents to patients or be inserted into the chambers comprising dual-function syringes of an apparatus, which will then is capable of compounding one or more of the bioactive agents into a composition to create personalized treatments for each patient.

In one aspect, the invention provides a system for preparing active agents for personalized treatment of a subject, wherein the system comprises a housing enclosing an interior space; an inventory structure comprising a plurality of chambers, wherein each chamber is capable of holding a dual-function syringe comprising a predetermined agent to be administered; means for selecting one or more dual-function syringes according to the predetermined active agent; means for moving the selected dual-function syringe to a desired location for transferring of the predetermined agent from the dual-function syringe to a product container or carrier; and means for sequentially transferring a controlled quantity of the predetermined agent from each selected dual-function syringe to the product container or carrier under positive or negative pressure.

This system and method for personalized injection treatment can be synchronized with a complementary health management system (a computerized and networkable via wireless or wired connection) and will automatically compound and record patient specific doses of injectable medicine for treatment and the patient's progress can be monitored on the Internet longitudinally. The subject herein can be a sick or healthy human or animal (e.g., mammals such as dogs, cats, pigs, horses or cattle). The term “bioactive agent” includes, but is not limited to biologics, vitamins, nutraceuticals, drugs, pharmaceuticals, or combinations thereof. The invention herein will compound the components with a customized, personalized dosage proportion, and then prepare a single product container which can be one of the following: a vial, dropper, patch, syringe, micro-injector, or another administration method. The size of the invention may be similar to those gourmet single cup coffee machines or inkjet/laser printers. The proportion of bioactive agents to be compounded is determined by the physician or by an Internet or computerized tool that is scientifically validated. Operators such as physicians or nurses may input a compounding order directly and manually by using a control pad. The compounding system itself may have a control screen such as a LED and LCD monitor, or some other external electronic system through which information about personalized care can be input. Alternately, the system can have a touch screen unit combining display and input units. When the compounded product is created, the optimizer will input the patient record into the server simultaneously. Some sort of label may be printed out to indicate that the compounded treatment is meant for the specific subject, and may indicate the different bioactive agents that are present in the final product. The patient may have received a set of codes in advance via an Internet tool and may bring in the print-out for the physician, who may then use the compounding machinery to deliver their product treatment. The inventory structure can have a plurality of vertical chambers individually holding one or more bioactive agent-containing dual-function syringes.

Each chamber can typically store 1 dual-function syringe, and there can be anywhere from about 1 to about 100 chambers. The dual-function syringes can have a volume capacity of about 0.001 mL to about 100.0 mL, and can be filled with liquid or solids. The bioactive agent-containing dual-function syringes and the chambers will be clearly labeled and color coded. Once the bioactive agent-containing dual-function syringes are loaded into the device, it will compound the exact proportion into a single product. This product can then be administered intravenously (IV), intramuscularly (IM), subcutaneously (SC), orally, topically, among other administration methods. The bioactive compounds are thoroughly sterilized and product containers are also sterilized and can be loaded by an operator. The dual-function syringes and the resulting product containers can be completely or partially filled with the bioactive agents.

Depending on the compounding order, the dual-function syringes can be selectively loaded and transported to a separate processing area. At the processing area or within the compounding machine, the selected dual-function syringes can be connected to the product container and a quantity of each predetermined bioactive agent can be transferred to the product container through a gravity-driven piston that is activated by an electromechanics or pneumatic (pressure-based) system. The positive pressure can be generated by mechanical compress on the plunger inside the syringe and the negative pressure inside the selected dual-function syringes can be employed by a vacuum pump in order to draw the bioactive agent out of the dual-function syringe. Alternatively, the dual-function syringes can be properly pressurized and packaged so that the bioactive agent may automatically come out without auxiliary pressurization. The transferring quantity can be controlled by regulating the applied pressure and time. The consumed dual-function syringes are disposed of automatically after single-use and can be disposed within the compounding machine itself or externally in a biohazard waste bin or sharps container.

The bioactive agent inside the dual-function syringes can be transferred through a connector, which is pre-assembled with a product vial as a cap before it is placed into the system and consisted of a needle and an air vent. The bioactive agents can also be discarded with the dual-function syringe or may be discarded by other methods that follow safety guidelines. Alternatively, the bioactive agent can be transferred through an extraction needle engaged with the loaded dual-function syringes and an injection system engaged with the product vial. The system may be enclosed by a housing and an air ventilation system may be included in the apparatus. All the embodiments described below can include sterilization means such as UV lamps to provide an internal aseptic environment, or some sort of washing mechanism.

A plurality of sensors such as proximity sensors, distance sensors, and temperature sensors may be placed in order to monitor if the desired dual-function syringes have been loaded, in order to monitor if the system operates properly, in order to check if the gate/door is closed, and also in order to monitor temperature inside the system. Sensors can be used in each of the embodiments described below and are commercially available, for example, from Sharp in Japan, Keyence Corporation of America in Itasca, Ill., Automation Direct in the U.S., and c3 controls in the U.S. After all the processes are completed, the product container can be manually taken out by an operator. Labels which include patient information and compounding order with text, bar code, or QR code format, can be printed out and the labels will be attached on the product container and the patient chart. This can then be administered to the patient in any administration method such as intravenously (IV), intramuscularly (IM), subcutaneously (SC), orally, topically, and through inhalation, to name a few.

The bioactive agents contained in the dual-function syringe may also be injected directly into the patient. Because the dual-function syringes are injection devices themselves, the bioactive agents may be injected straight into patients by administering external pressure to the plunger. Alternatively, 1 to 100 of these dual-function syringes can be stored in the chambers of the compounding machine, which will be filled with pre-determined amounts of each bioactive agent to create the personalized treatment dosages. The dual-function syringes can have a volume capacity of about 0.001 ml to about 100.0 ml. The prefilled dual-function syringes will be clearly labeled, and each will be color-coded. The dual-function syringes can be completely or partially filled with the bioactive agent. Once the dual-function syringe is loaded in the chamber, a quantity of the predetermined bioactive agent can be transferred to the product container through gravity driven methods such as mechanical extrusion or pneumatic (pressure driven) systems.

Alternatively, the dual-function syringe can be properly pressurized and packaged so that the solution may automatically come out without external pressurization, and the transferring quantity can be controlled by regulating the amount of applied pressure and for how long. The dual-function syringe is then disposed after a single use. The bioactive agent can also be disposed.

In some embodiments, the system can be used in combination with alternate technological systems or devices to allow communication specifying what patients will individually need from their treatment, and lead to the formation of individually compounded mixtures of bioactive agents.

In some embodiments, the system can print labels and have color-coded chambers and dual-function syringes to allow for the organization of both the bioactive agents, and the resulting treatments.

In one aspect, the present invention provides an automated system for compounding bioactive agents for treatment of a patient is provided herein. In some embodiments, the system comprises: a housing enclosing an interior space; an inventory structure having a plurality of chambers for individually holding a dual-function syringe, wherein each dual-function syringe holds has a volume capacity from about 0.001 mL to about 100.0 mL; means for selecting dual-function syringes in accordance with predetermined bioactive agents contained in said dual-function syringes; means for moving the selected dual-function syringes to a processing area or to the compounding machine or directly administering to the patient; means for sequentially transferring a controlled quantity of the predetermined bioactive agent from each selected dual-function syringe to a product container such as, but not limited to, a vial, syringe, dropper, patch, or micro-injector through electro-mechanical or pressure-driven pneumatic systems; and means for automatically discarding spent dual-function syringes from which the bioactive agents have been removed after a single use.

In some embodiments, the dual-function syringe itself will be a system for holding and injecting active agents, and can be used in conjunction with the aforementioned automated system to combine these agents to create personalized treatments. In some embodiments, the dual-function syringe system comprises: a syringe holder locking in the syringe, a needle rubber between the syringe holder and the syringe, a syringe which has a volume capacity from 0.001 mL to 100.0 mL liquid and will contain a predetermined pharmaceutical agent, a plunger within the syringe, a needle and needle cap screwed to the bottom of the syringe, and a holder cap; means for moving the selected systems to the patient or to an auxiliary machine; means for administering the contained bioactive agent; and means for discarding the dual-function syringe system after a single use.

This innovative technology will enable a novel personalized medicine platform and it can be used to safely and efficiently to expand physician services and improve treatment accuracy and its outcomes. This device is applicable for patients requiring effective doses of treatment combinations of any bioactive agents, but can also be utilized by patients needing just one specific bioactive agent.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1 is a 3D rendering of basic layout of the automated compounding system which identifies and illustrates the position of different components of the system such as the LCD screen, piston, chambers and vial. The dual-function syringes are housed in the chambers.

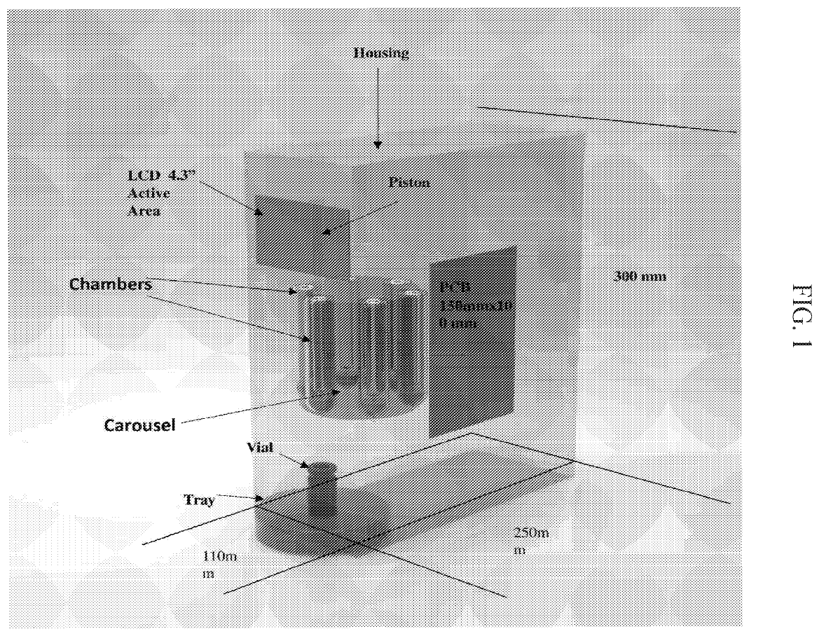

FIG. 2 is a view of a chamber holder layout which specifies the parts piston, rubber, holder, syringe, needle and color label.

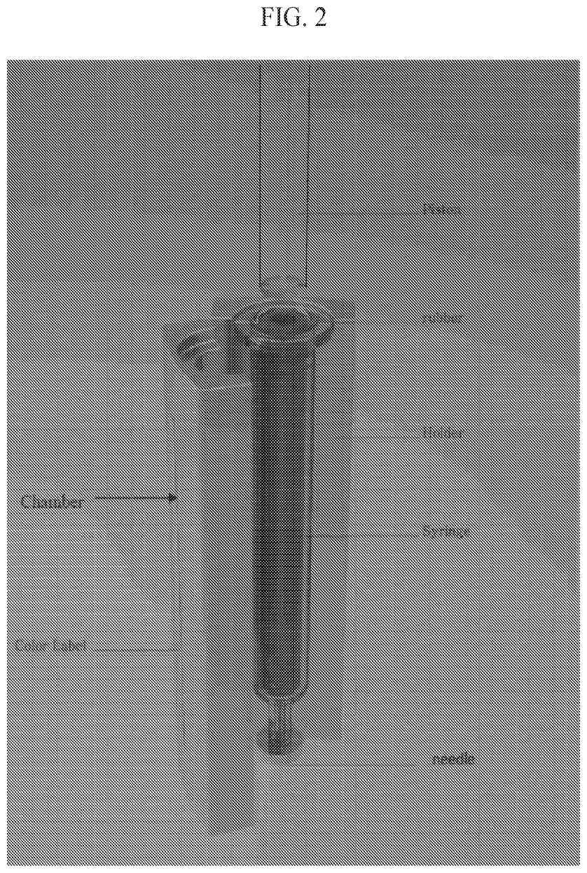

FIG. 3 is a view of the dual-function syringe system.

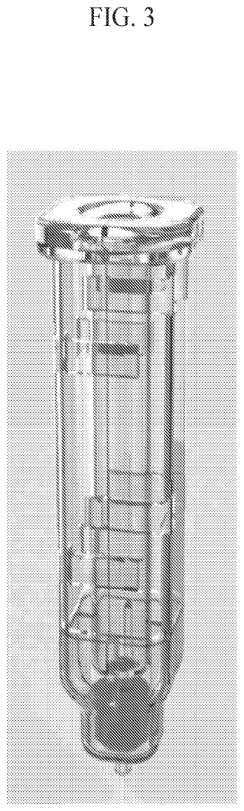

FIG. 4 is a view of the dual-function syringe system explaining different parts of the system namely the holder cap, syringe, needle rubber and syringe holder. It depicts the modularity of the system.

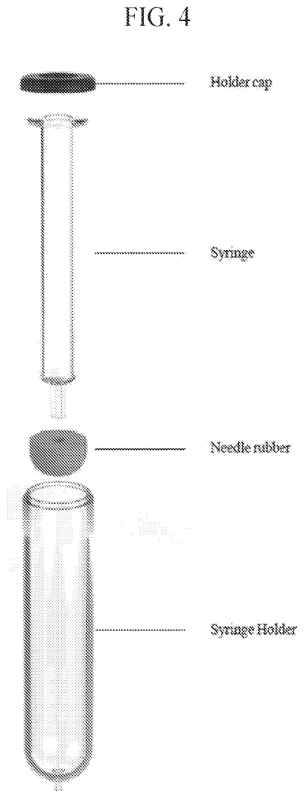

FIG. 5 shows a view of an assembled dual-function syringe system.

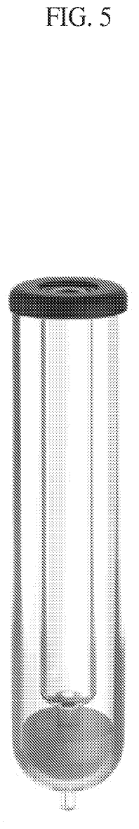

FIG. 6 shows a view of an assembled dual-function syringe system.

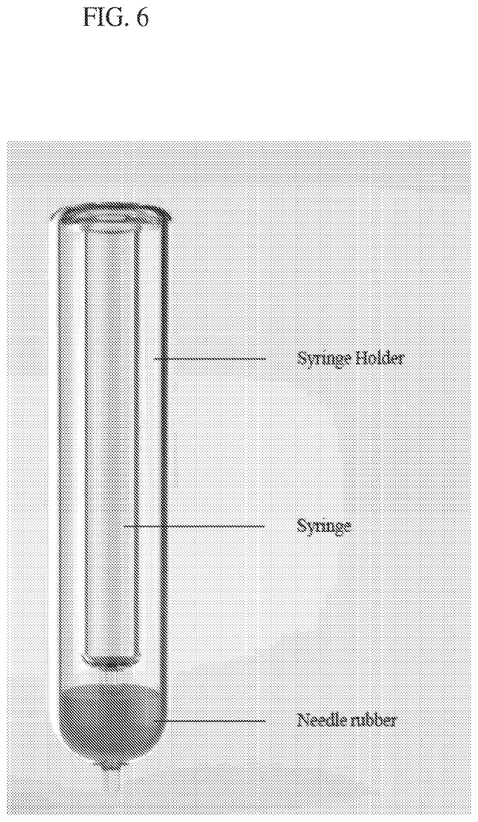

FIG. 7 is a view of the assembled dual-function syringe system with a needle tip attached.

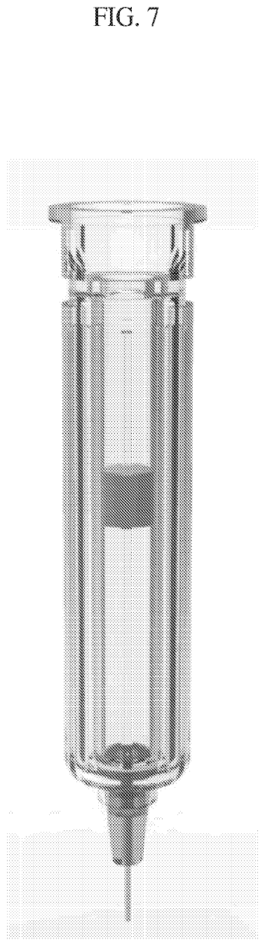

FIG. 8 is a view of the dual-function syringe system explaining different parts namely the syringe, needle and holder. It depicts the modularity of the system.

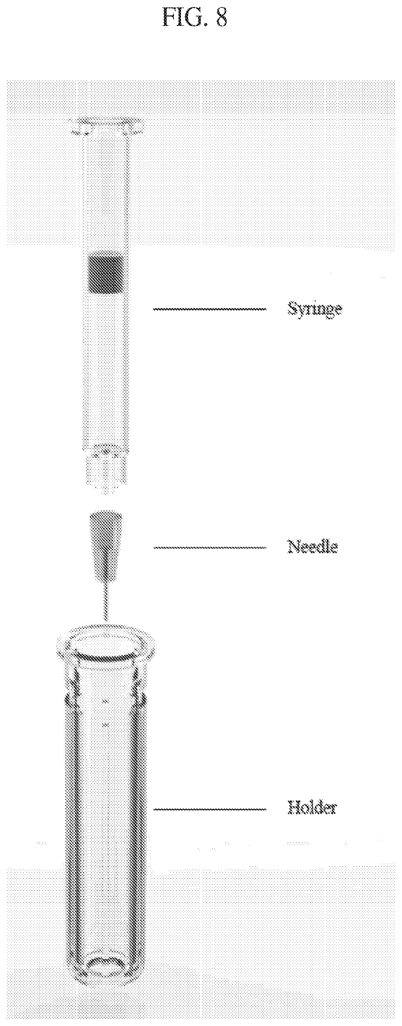

FIG. 9 shows a housing apparatus for the dual-function syringe system.

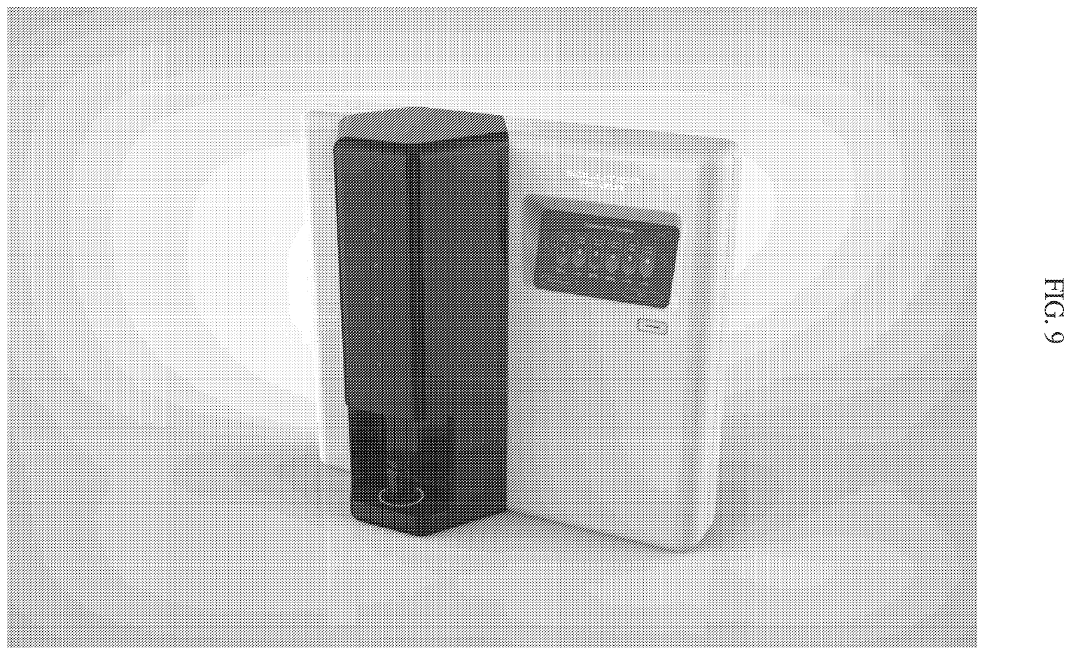

FIG. 10 shows a housing apparatus for the dual-function syringe system.

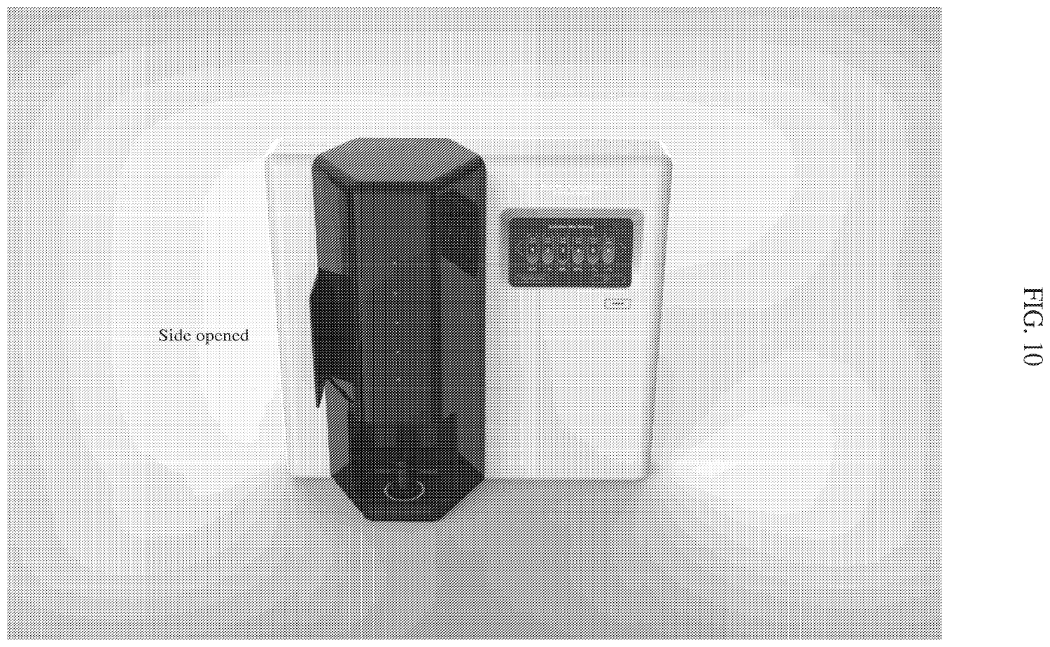

FIG. 11 shows a housing apparatus for the dual-function syringe system.

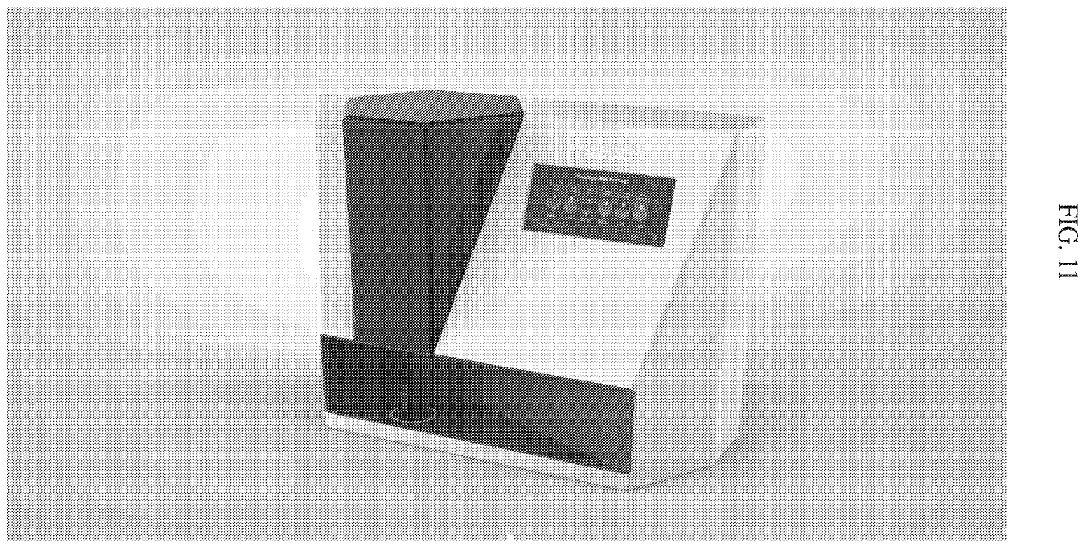

FIG. 12 shows a housing apparatus for the dual-function syringe system.

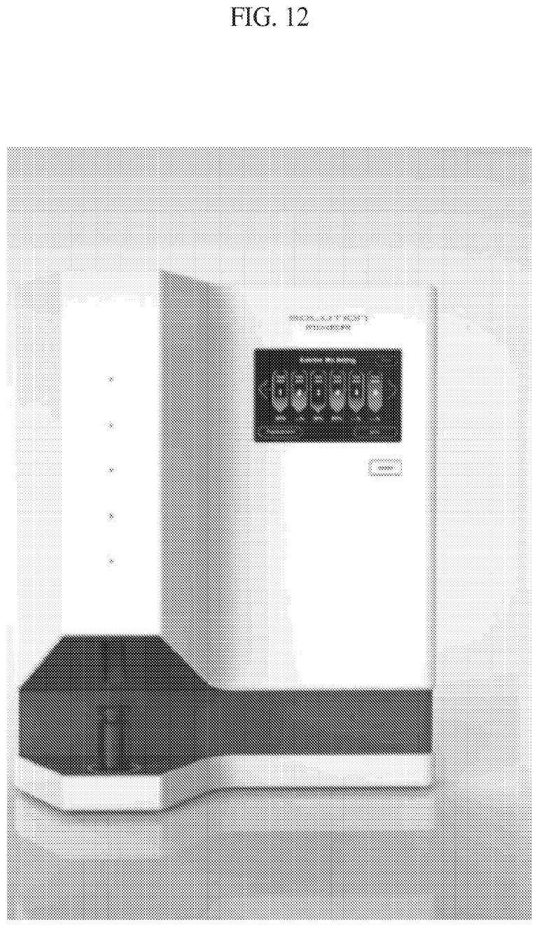

FIG. 13 shows a housing apparatus for the dual-function syringe system. Part 1 depicts a front cover. Part 2 depicts a rear cover. Part 3 depicts a side cover. Part 4 depicts an inner cover. Part 5 depicts an LCD window. Part 6 depicts a power button. Part 7 depicts a vial ring LED.

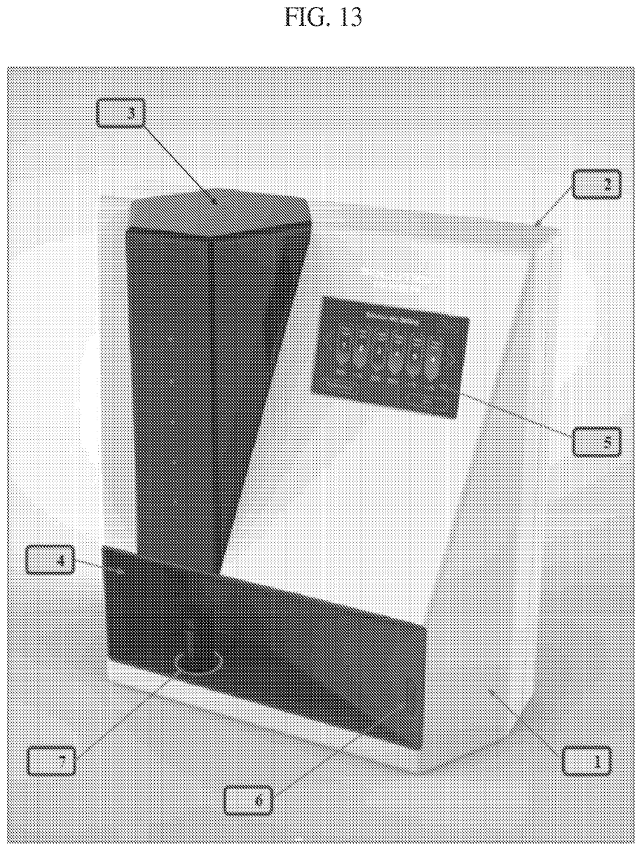

FIG. 14 shows a microneedle device which can be used in the system of the invention. The vial of the microneedle device can be used as a product container to capture agents released from the dual-function syringe system. A) microneedles B) head piece C) device reservoir.

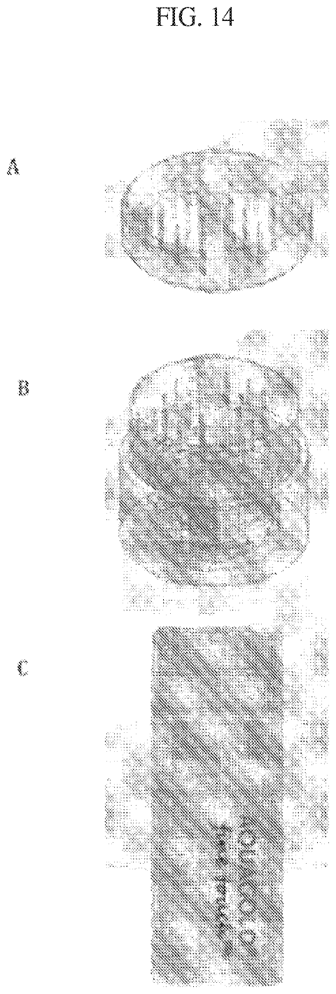

FIG. 15 shows a microneedle device which can be used in the system of the invention. The vial of the microneedle device can be used as a product container to capture agents released from the dual-function syringe system.

### DETAILED DESCRIPTION

The present invention relates to a system and method for compounding bioactive agents, which will utilize dual-function syringes to transfer these agents while also being capable of acting as a stand-alone instrument that helps in administration of drugs to subjects.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 2%, 3%, 4%, or 5% of a particular term.

Other than in the working examples or where otherwise indicated, all numbers expressing amounts and volumes of materials, reaction conditions, time durations, quantified properties of materials, and so forth, stated in the specification and claims are to be understood as being modified in all instances by the term “about.”

It will also be understood that any numerical range recited herein is intended to include all sub-ranges within that range.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

In one embodiment, the invention comprises a system for preparing active agents for personalized treatment of a subject, wherein the system comprises
i) a housing enclosing an interior space;
ii) an inventory structure comprising a plurality of chambers, wherein the chamber is capable of holding a dual-function syringe comprising a predetermined agent to be administered;
iii) means for selecting one or more dual-function syringes according to the predetermined active agent;
iv) means for moving the selected dual-function syringe to a desired location for transferring of the predetermined agent from the dual-function syringe to a product container or carrier; and
v) means for sequentially transferring a controlled quantity of the predetermined agent from each selected dual-function syringe to the product container or carrier under positive or negative pressure.

In some embodiments, the system further comprises means for automatically discarding dual-function syringes from which the agents have been released. In some embodiments, a used dual-function syringes can be disposed of within the compounding machine or manually discarded. In some embodiments, the means for discarding the used dual-function syringe comprises a waste bin positioned in the interior of the housing into which the used dual-function syringes are dropped. In some embodiments, the means for discarding the used dual-function syringes comprises manual transport to a waste bin.

In some embodiments, the means for disposing waste produced by cleaning of the dual-function syringe injection system comprises a drain leading to a waste solution container within the interior of the housing. In some embodiments, means for cleaning the injection system includes a dual-function syringe containing a sterile cleaning solution comprising ethanol and/or deionized water. In some embodiments, the system further comprises a UV sterilizing lamp positioned in the interior of the housing.

In some embodiments, the system further comprises a pressure/vacuum pump.

In some embodiments, the system is configured to limit the dual-function syringes to a single use. In some embodiments, the syringe is discarded after a single use. In some embodiments, the needle head breaks after the first administration or extrusion rendering the dual-function syringe not reusable.

In some embodiments, the means for transferring the agent comprises a means for mechanically compressing the dual-function syringe to provide a positive external pressure for forcing the agent from the dual-function syringe through a needle and into the product container or carrier. In some embodiments, the means for transferring the agent comprises administering external pressure on a plunger. In some embodiments, the means for transferring the agent comprises grasping and pulling the plunger of a hypodermic syringe, and further includes means for removing the cap. In some embodiments, at the processing area or within the compounding machine, the selected dual-function syringes can be connected to the product container and a quantity of each predetermined bioactive agent can be transferred to the product container through a gravity-driven piston that is activated by an electromechanics or pneumatic (pressure-based) system.

In some embodiments, the means for moving the selected dual-function syringes to a desired location comprises a manual transport system. In some embodiments, the means for moving is using an electro-mechanical system.

In some embodiments, the system can be used to inject an agent directly into a patient. In some embodiments, the product container or carrier is selected from a receiving vessel, container, microinjection device, vial, microneedle device, syringe, and a patch for topical treatment to a subject's skin, a dropper capable of applying treatment to skin, container capable of condensing powder into an orally administered capsule or tablet, and a container capable of holding liquid which can be nasally inhaled.

In some embodiments, the inventory structure comprises 2 to about 100 chambers that house the syringes. In some embodiments, the system comprises 2 to about 50 chambers that house the syringes. In some embodiments, the system comprises 2 to about 25 chambers that house the syringes. In some embodiments, the system comprises 2 to about 10 chambers that house the syringes. In some embodiments, one or more chambers comprise a dual-function syringe comprising a powder or gel that is transferred from the dual-function syringe without a needle or needle tip to enable passage of the powder or gel through the dual-function syringe. In some embodiments, the dual-function syringes are placed in the chambers, and then disposed of within the inventory structure or removed from the inventory structure. In some embodiments, the inventory structure comprises a carousel, wherein the chambers are arranged around a circumferential periphery of the carousel. In some embodiments, one or more of the chambers are pre-assigned to dual-function syringes with specific active agents. In some embodiments, at least one of the chambers is capable of being personalized based on the needs of the patient. In some embodiments, the personalized chamber comprises a syringe holder, capable of receiving a syringe comprising an agent suitable for the patient. In some embodiments, the chambers and dual-function syringes are color coordinated.

In some embodiments, the dual function syringe is shown in the accompanying Figures herein. In some embodiments, each dual-function syringe comprises a syringe holder locking into place a syringe, optionally with a removable holder cap which covers the syringe; a needle rubber stopper cushioning the area between the syringe holder and the syringe; a hypodermic syringe which has a volume capacity from about 0.001 mL to about 100 mL; a plunger within the syringe; optionally a removable metal needle; optionally a needle tip screwed on to the bottom of the syringe; and optionally a removable holder cap which covers the syringe.

In some embodiments, the dual-function syringe has a volume capacity of from about 0.1 to about 100 mL liquid. In some embodiments, the volume capacity is about 0.1 to about 40.0 mL liquid, about 0.1 to about 30 mL liquid, about 0.1 to about 20 mL liquid, or about 0.1 to about 10.0 mL liquid.

In some embodiments, the dual-function syringes have an elongated cylindrical or prolate configuration and are horizontally or vertically oriented in respective chambers.

In some embodiments, the dual-function syringe can be attached to a needle. In some embodiments, the dual-function syringe can be attached to a microneedle capable of administering liquids and/or viscous solutions.

In some embodiments, the dual-function syringe comprises a circular cylindrical syringe holder, a hypodermic syringe which may or may not have a needle and needle tip screwed onto the bottom, a rubber stopper between the syringe and syringe holder, a rubber plunger within the syringe with which the agent can be transferred by providing positive external pressure, a holder cap covering the top of the syringe, and a means for pushing the plunger down.

In some embodiments, the syringe holder has a divert around its circumference or some other attachment mechanism which firmly locks the syringe in place as the agent is contained and administered. In some embodiments, the syringe holder and the syringe are clear glass.

In some embodiments, the dual-function syringes can be provided packaged in a box containing from 1 to about 50 dual-function syringes.

In some embodiments, the system comprises a locking mechanism which attaches the syringe to the syringe holder, locking it into place.

In some embodiments, the system further comprises an injection system positioned within the housing, the injection system comprising an injection nozzle which is movable between an initial upper position and a lower position for insertion into a product container or carrier. In some embodiments, the injection nozzle is also laterally movable. In some embodiments, the system further comprises a vacuum conduit associated with the injection nozzle.

In some embodiments, the system further comprises a plurality of sensors positioned in the interior of the housing for monitoring the active agent inventory and operation of the system.

In some embodiments, the system further comprises a label printer.

In some embodiments, the system further comprises information input and output units.

In some embodiments, the system is in communication with a computer. In some embodiments, the computer accesses patient or health related information. In some embodiments, the system is in communication with a portable electronic device. In some embodiments, the computer or portable electronic device is capable of operating the system. In some embodiments, the system is capable of being operated remotely by a user.

In some embodiments, the system can be attached to Smart Label Data Transmission Systems as disclosed in U.S. Appl. Pub. No.: 2014/0266581, which are applied for end-user optimization. This allows for both customization for both the device and for the patient, and monitoring for counterfeit medicine.

The predetermined agent can be in any form. In some embodiments, it is in liquid, solid or gel form. In some embodiments, the system compounds a plurality of active agents into a single composition for administration to a subject in need of treatment. In some embodiments, the predetermined agents comprise one or more of bioactive agents, topical creams, nasal sprays, injectables, and pharmaceutical compounds.

In some embodiments, the dual-function syringes contain the same bioactive compound, different bioactive compounds, or are empty.

In some embodiments, the one or more of the chambers comprises a dual-function syringe comprising one or more vitamin compounds or compositions as described in U.S. Patent Appl. Pub. No. 2015/0238527, which is herein incorporated by reference in its entirety. In some embodiments, the system compounds a composition comprising a plurality of vitamins. In some embodiments, the vitamins are compounded to be administered orally in solid or liquid form or by injection in liquid form.

In some embodiments, the one or more of the chambers comprises a dual-function syringe comprising one or more vitamin compounds or compositions as described in U.S. Patent Appl. Pub. No. 2016/0175408, which is herein incorporated by reference in its entirety.

In some embodiments, one or more of the chambers comprises a dual-function syringe comprising one or more cannabinoid compounds. In some embodiments, the cannabinoid compound comprises THC and/or CBD, analogs or derivatives thereof. In some embodiments, the THC and CBD is mixed in specific ratios as prescribed for medical treatment.

In some embodiments, one or more of the chambers comprises a dual-function syringe comprising one or more active agents selected from hyaluronic acid (e.g., cross-linked, non-crosslinked, or a combination thereof) neuromodulator, stem cells, an antibody, a biologic, a small molecule therapeutic, an antigen, botulinum toxin (e.g., botulinum toxin of serotype A, B, C, D, E, F or G), a vitamin, a mineral, PLLA (poly-L-lactic acid), platelet-rich plasma, retinol, retinoic acid, a bleaching/whitening agent, collagen, a neurotoxin, an anesthetic or any combination thereof.

The one or more agents that can be employed in the syringes is not limiting.

In some embodiments, the active agent is hyaluronic acid. Hyaluronic acid and sodium hyaluronate can be used interchangeably herein. The hyaluronic acid (HA) is involved in cartilage resilience and skin repair, has been applied medically for decades for a number of different uses including, for example, cartilage resilience and skin repair. Among the most common of these medical applications employ injectable delivery, for example to treat joint pain, or topical delivery, for example to treat dermatitis. Cosmetically, it is often used as an active agent in facial filler injections to smooth wrinkles and in topical creams and gels to rejuvenate the skin and combat the aging process. Hyaluronic acid includes both cross-linked and non-cross-linked hyaluronic acids.

Micro Hyaluronic Acid FCH is highly stable against heat and pH change, exhibits great permeability to stratum corneum and excellent moisturizing property, and can be used as a cosmetic ingredient. Micro Hyaluronic Acid FCH fits comfortably in skin and has excellent permeability to the stratum corneum. Micro Hyaluronic Acid FCH is ultra low viscosity sodium hyaluronate which has an average molecular weight of under 5000. Micro Hyaluronate Acid FCH permeates into skin and shows excellent moisturizing property.

In some embodiments, the hyaluronic acid is crosslinked. In some embodiments, the hyaluronic acid is non-crosslinked. In some embodiments, at least a portion of the hyaluronic acid is crosslinked.

In some embodiments, the one or more bioactive agents is elected from the group consisting of one or more vitamins, one or more minerals, retinol, retinoic acid, a bleaching/whitening agent, collagen, a neuromodulator, poly-L-lactic acid, an anesthetic and combinations thereof. In some embodiments, the neuromodulator comprises botulinum toxin (e.g., botulinum toxin of serotype A, B, C, D, E, F or G).

In some embodiments, the one or more bioactive agents that can be included is discussed below.

Vitamins, or vital nutrients, and minerals are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium. When referring to a vitamin, it would be understood that all chemical forms of the vitamin are contemplated.

B vitamins are a group of water-soluble vitamins that play important roles in cell metabolism. The B vitamins are B1 (thiamine), B2 (riboflavin), B3 (niacin), B5 (pantothenic acid), B6 (pyridoxine), B7 (biotin), B12 (cobalamin) and folic acid. The B vitamins play an important role in many aspects of the body's functioning, and a vitamin B deficiency can have a serious impact on overall health.

Vitamin B supplements are known in the art: such formulations are limited in terms of absorption (oral dosage forms) or may require a hospital visit (IV therapy) at significant cost in terms of time and expense.

Collagen is a type of fibrous protein found most often in the skin, flesh, and connective tissue of vertebrates. In mammals, it is the most abundant protein in the body, and provides structural support for major tissues and organs. In the skin, it is responsible for providing structure, firmness, and smoothness, and it is often a decrease in collagen production that leads to chronic aging. For this reason, collagen is often injected or topically introduced to the skin in attempts to slow or reverse the effects of aging.

Vitamins and minerals, or vital nutrients, are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium.

Vitamin B12, also called cobalamin, is a water-soluble vitamin with a key role in the normal functioning of the brain and nervous system, and for the formation of blood. It is one of the eight B vitamins. It may be involved in the metabolism of every cell of the human body, especially affecting DNA synthesis and regulation, but also fatty acid synthesis and energy production. Vitamin B12 may also be involved in maintenance of the central nervous system and has been used to affect memory loss, Alzheimer's disease, boosting mood, energy and concentration, boost the immune system, and slow aging. Vitamin B12 may also play a role in heart disease, lowering high homocysteine levels (which may contribute to heart disease), male infertility, diabetes, sleep disorders, depression, mental disorders, weak bones (osteoporosis), swollen tendons, AIDS, inflammatory bowel disease, asthma, allergies, a skin disease called vitiligo, preventing cervical and other cancers, and skin infections. Two common forms of Vitamin B12 are cyanocobalamin and methylcobalamin.

Vitamin B12 deficiency may cause macrocytic anemia, fatigue, loss of appetite, loss of balance, weakness, and mood disturbances. It also may cause serious neurologic and neuropsychiatric illness such as paresthesias, ataxia, and memory loss. Vitamin B12 absorption may be impaired at the level of the stomach, where intrinsic factor is produced, or at the level of the terminal ileum, where intrinsic factor bound to vitamin B12 is absorbed.

Niacin and nicotinamide, also known as niacinamide, are forms of vitamin B3. Nicotinamide is the amide of nicotinic acid (vitamin B3/niacin). Nicotinamide is a water-soluble vitamin and is part of the vitamin B group. Nicotinamide may be used for preventing vitamin B3 deficiency and related conditions such as pellagra. Each of these forms of vitamin B3 may be used for schizophrenia, hallucinations due to drugs, Alzheimer's disease and age-related loss of thinking skills, chronic brain syndrome, depression, motion sickness, alcohol dependence, and fluid collection (edema).

Vitamin B1, also known as thiamine, is a water-soluble vitamin and may be utilized for metabolizing carbohydrates and production of energy. Vitamin B1 also may aid in the function of the heart and cardiovascular system and the nervous system.

Vitamin B6, also known as pyridoxine, may be involved in many aspects of macronutrient metabolism, neurotransmitter synthesis, histamine synthesis, hemoglobin synthesis and function and gene expression. Vitamin B6 may assist with cellular metabolism, supports the immune system, with formation of red blood cells and maintenance of healthy brain function. Vitamin B6 may be used for Alzheimer's disease, attention deficit-hyperactivity disorder (ADHD), Down syndrome, autism, diabetes and related nerve pain, sickle cell anemia, migraine headaches, asthma, carpal tunnel syndrome, night leg cramps, muscle cramps, arthritis, allergies, acne and various other skin conditions, and infertility. It is also may be used to treat dizziness, motion sickness, preventing the eye disease age-related macular degeneration (AMD), seizures, convulsions due to fever, and movement disorders (tardive dyskinesia, hyperkinesis, chorea), as well as for increasing appetite and helping people remember dreams. Vitamin B6 may be used for acne, leprosy, attention deficit-hyperactivity disorder (ADHD), memory loss, arthritis, preventing premenstrual headache, improving digestion, protecting against toxins and pollutants, reducing the effects of aging, lowering blood pressure, improving circulation, promoting relaxation, improving orgasm, and preventing cataracts. Vitamin B6 deficiency may cause anemia due to insufficient production of hemoglobin.

Vitamin B2, also known as riboflavin, releases energy from carbohydrates and may be used for preventing low levels of riboflavin (riboflavin deficiency), cervical cancer, and migraine headaches. It also may be used for treating riboflavin deficiency, acne, muscle cramps, burning feet syndrome, carpal tunnel syndrome, and blood disorders such as congenital methemoglobinemia and red blood cell aplasia. It also may be used for increasing energy levels; boosting immune system function; maintaining healthy hair, skin, mucous membranes, and nails; slowing aging; boosting athletic performance; promoting healthy reproductive function; canker sores; memory loss, including Alzheimer's disease; ulcers; burns; alcoholism; liver disease; sickle cell anemia; and treating lactic acidosis brought on by treatment with a class of AIDS medications called NRTI drugs.

The term “vitamin B6” encompasses multiple forms of vitamin B6 suitable for human administration. Several forms of the vitamin are known, but pyridoxal phosphate (PLP; “pyridoxine”) is the active form and may be used as a cofactor in many reactions of amino acid metabolism, including transamination, deamination, and decarboxylation. Pyridoxine may be used in enzymatic reactions affecting the release of glucose from glycogen.

Vitamin C, also known as ascorbic acid, is an antioxidant. Vitamin C may be used to protect against free radicals and promote a healthy immune system, wound healing, and forming healthy skin. More specifically, ascorbic acid may be used to prevent and treat scurvy, a disease caused by a lack of vitamin C in the body. People with high intakes of vitamin C from fruits and vegetables may have a lower risk of getting many types of cancer, such as lung, breast, and colon cancer.

Vitamin B5, also known as pantothenic acid, has skincare benefits. For example, it increases the degree of hydration of the skin, reduces the trans-epidermal water loss and keeps the elasticity and smoothness of the skin. Vitamin B5 may be used in acne treatments and may be used to reduce itchiness of the skin.

Zinc is an essential mineral found in cells throughout the body. Zinc is required for protein synthesis and collagen formation, and may be used to promote a healthy immune system and assist in wound healing. It may also be used for muscular growth and contraction and to protect the liver from chemical damage such as which can occur with anesthetics or other drugs or toxins. Zinc may also be utilized in bone formation. Zinc deficiency may contribute to fatigue, susceptibility to infection, and slow wound healing.

Ahseutasanchin is a unique pigment belonging to the carotenoid family. It exhibits antioxidant capacity against free radicals.

Vitamin E can boost the immune system and protect people against toxins such as air pollution, neurological disease such as Alzheimer's disease, and diabetes. As an antioxidant, Vitamin E can remove free radicals that damage the cell structure. Owing to this property, another well-known health benefit for Vitamin E is in skin and hair care.

Vitamin D helps intestines absorb nutrients and is essential for calcium utilization, ensuring strong bones and robust immune system.

Selenium displays antioxidant properties that regenerate vitamin C and vitamin E, thereby decreasing the aging of the skin and protecting cells from damage. Moreover, selenium also benefits the immune system and protects our body against various infections.

Glutathione(GSH) is an antioxidant which prevents damage to important cellular components caused by free radicals. The strong antioxidant effect of glutathione helps keep cells running smoothly and it also helps the liver remove chemicals that are foreign to the body, such as drugs and pollutants.

Anthocyanidins have a wide range of biological activities including antioxidant, anti-inflammatory, antimicrobial and anti-cancer activities. In addition they display a variety of effects on blood vessels, platelets and lipoproteins able to reduce the risk of coronary heart diseases.

EPA is a form of omega-3 fatty acids which can reduce cellular inflammation.

DHA is a building block of tissue in the brain and retina of the eye. It helps with forming neural transmitters, such as phosphatidylserine, which is important for brain function. EPA and DHA are also well-known for improving skin conditions. Their anti-inflammatory properties help prevent various skin ailments. EPA and DHA can also reduce the damage caused by overexposure to the sun and negative impacts of UV rays.

Lecithin acts as a solvent for cholesterol, triglycerides, and other fats. Therefore, it helps to prevent such ailments as high blood pressure, stroke, heart disease, hardening of the arteries, etc. Also, lecithin plays a vital role in the absorption of nutrients out of the blood stream into the cells.

CoQ10 helps to combat fatigue, boosts immune system, fight against free radicals, and keep cells both inside the body and in the skin healthy. The CoQ10 level decreases as people get older, resulting in an impeded ability to produce collagen and elastin, and the loss of collagen and elastin causes our skin wrinkle and sag.

Magnesium can benefit blood pressure and help prevent sudden cardiac arrest and stroke. It also plays a role in detoxification processes and therefore is important for helping to prevent damage from environmental chemicals, heavy metals and other toxins.

Vitamins and minerals, or vital nutrients, are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium.

Bleaching/whitening agents that may be used in the compositions described herein include, but are not limited to, hydroquinone, kojic acid, ascorbic acid, magnesium ascorbyl phosphate or ascorbyl glucosamine, hydroquinone, licorice extract (e.g., Glycyrrhiza Glabra (licorice) root extract), an alpha MSH antagonist (e.g. undecylenoyl phenylalanine), phytic acid, monobenzyl ether of hydroquinone, azelaic acid, kojic acid, mequinol, retinoids (e.g., tretinoin, adapalene), soy proteins, alpha-hydroxy acids (e.g., glycolic acid), trichloroacetic acid, salicylic acid, hydroquinone-beta-D-glucopyranoside, paper mulberry, glabridin, 4-isopropylcetchol, aleosin, N-acetyl-4-S-cycteaminylphenol, N-propionyl-4-S-cysteaminylphenol, N-acetyl glucosamine, tranexaminc acid and mixtures thereof.

Retinols used in the compositions described herein include, but are not limited to, retinoic acid.

Collagen is a type of fibrous protein found most often in the skin, flesh, and connective tissue of vertebrates. In mammals, it is the most abundant protein in the body, and provides structural support for major tissues and organs. In the skin, it is responsible for providing structure, firmness, and smoothness, and it is often a decrease in collagen production that leads to chronic aging. For this reason, collagen is often injected or topically introduced to the skin in attempts to slow or reverse the effects of aging (Varani J, Dame M K, Rittie L, Fligiel S E G, Kang S, Fisher G J, Voorhees J J: Decreased Collagen Production in Chronically Aged Skin. Am J Pathol. 2006 June; 168(6):1861-1868. PubMed PMCID: PMC1606623).

Botulinum toxin, a neurotoxic protein, is used cosmetically and therapeutically for treatment of facial lines and wrinkles, upper motor neuron syndrome, excessive sweating, cervical dystonia, chronic migraine, and overactive bladder. The toxin is generally injected into the subcutaneous muscles at the target areas, and works by temporarily (for a period of six weeks to eight months, depending on the location and the dose) inhibiting the release of acetylcholine at the neuromuscular junction and thus paralyzing the muscles achieve the desired affects (BOTOX (onabotulinumtoxinA) [prescribing information] Irvine, Calif. Allergan, Inc. January 2013). Botulinum toxin refers to any botulinum toxin, including but not limited to botulinum toxin type A, botulinum toxin type B, botulinum toxin type Cl, botulinum toxin type D, botulinum toxin type E, botulinum toxin type F and botulinum toxin type G. Botulinum toxin type A includes, for example, Botox, Dysport and Xeomin. Botulinum toxin type B includes, for example, MyoBloc. Botulinum toxin may be provided in a liquid or powder form. A powdered form may be, for example, a sterile, lyophilized preparation. Lyophilized preparations may be reconstituted prior to application. Alternatively, botulinum toxin may be provided as a sterilized pre-dissolved solution. Botulinum toxin may be formulated in an amount of about 0.01 to about 60 units.

“MicroBotox” or “Purtox” as used herein, refers to instances when diluted Botox is injected in multiple very small doses in a treated area. The effects of the Botox are more evenly spread over the areas treated and the risks of having areas over-treated is reduced. Use of MicroBotox generally results in a more natural look (i.e., less frozen) and the dosage of Botox administered is reduced. For some patients suffering from recalcitrant acne problems, MicroBotox (referred to as “mesoBotox” when used in this situation) can be injected very superficially into the facial skin. Following dilution, microBotox may be formulated in an amount of 0.1 to about 99% of the compositions. For example, one would use 0.1 to about 100 units of onabotulinum toxin diluted with at least 2.5 cc of saline.

Minoxidil is a vasodilator that was originally administered orally as a treatment for hypertension, but was found to have the additional effect of slowing hair loss and promoting hair growth. It is now a common topical treatment for androgenic hair loss, and is thought to achieve hair regrowth by increasing the blood flow (and thus the availability of oxygen and vital nutrients) to the hair follicles, stimulating them to resume normal functioning (Olsen E A, Whiting D, Bergfeld W, Miller J, Hordinsky M, Wanser R, Zhang P, Kohut B: A multicenter, randomized, placebo-controlled, double-blind clinical trial of a novel formulation of 5% minoxidil topical foam versus placebo in the treatment of androgenetic alopecia in men. J Am Acad Dermatol. 2007 Aug. 29. PubMed PMID: 17761356).

Platelet-rich plasma (PRP) is blood plasma that has been enriched by platelets, and is prepared by separating whole blood via centrifugation and then collecting the plasma-rich layers that emerge. Because it has five times the baseline platelet concentration of plasma (.about.100,000 platelets per microliter as opposed to the baseline of .about.20,000 platelets per microliter), it contains a number of different growth factors (proteins that stimulate tissue growth, the release of which can be induced by the addition of thrombin and calcium chloride. PRP injections have been used clinically for several years as a treatment for nerve, bone, and muscle injuries, and have been used cosmetically to reverse damage to the skin and to promote dermal strength and rejuvenation (Borrione P, Gianfrancesco A D, Pereira M T, Pigozzi F: Platelet-rich plasma in muscle healing. Am J Phys Med Rehabil. 2010 October; 89(10):854-61. PubMed PMID: 20855985).

Poly-L-lactic Acid (PLLA) is a type of dermal filler used in the treatment of facial lipoatrophy (the gradual loss of facial fat, generally due to aging). PLLA, upon entering the skin, provides immediate structural support to the skin and also promotes the neo-synthesis of collagen, hiding sunken areas. Over time, it is converted by the body into harmless lactic acid, gradually transferring the load to the recently synthesized collagen (SCULPTRA Aesthetic (injectable poly-L-lactic acid) [prescribing information]. Bridgewater, N.J. Sanofi-Aventis U.S. LLC. May 2012).

Bimatoprost is a prostaglandin prodrug that is administered topically to control the progression of Glaucoma and to treat ocular hypertension. Since 2008, the application of this drug has evolved to encompass a cosmetic formulation for the lengthening and darkening of eyelashes and is thought to confer an improved appearance by delivering bimatoprost—a growth stimulating analog—circambient to the hair follicles at the edge of the eyelid.

Compositions described herein may be used to treat one or more clinical manifestations and/or symptoms of a disease, such as skin conditions, hair loss, wound healing and/or prevention of scarring, or for anti-aging, longevity and wellness purposes described herein. The compounded compositions can be injected into a patient to improve skin elasticity, skin regeneration, metabolism, smoothness and/or softness of skin (i.e., making the skin feel smoother and softer following treatment); the overall appearance of skin; evening out skin tone and texture; clarity and/or radiance of skin; making the skin look younger; and making wrinkles appear softer and/or less prominent, etc.

In some embodiments, the compositions and methods provided herein provide compositions formulated for administration to the skin surface in combination with a microneedle device, the compositions being applied topically, intradermally or a combination of both routes of administration. The compositions and methods provided herein provide vitamin compositions formulated for administration to the skin surface in combination with a microneedle device, the compositions being applied percutaneously. Suitable amounts can be determined by the skilled physician and depend on the age, gender and general health of the patient, as well as a surgical, or other procedure planned for the patient. In some cases, it may also be determined based upon the degree and type of trauma a patient has suffered. Previous nutritional status may also be considered when determining a suitable amount of nutritional supplementation. In addition, patient compliance is a very important factor, since nutritional supplementation is not effective if the patient does not receive the supplements and is much less effective if the patient does not receive the proper dose on a consistent basis.

Improved skin health or improved skin quality, as used above, includes but is not limited to improved appearance, increased regeneration, increased elasticity, increased anti-oxidative level, reduced photo-aging, reduced wrinkles, reduced scarring, reduced bacterial activities including acne, redistribution of fat and/or other content of the skin, reduced number and/or sizes of pores, reduced callusing, reduced sweating and/or body odor through skin, improved scalp health, increased hair density, increased evenness of hair growth, and increased hair strength.

The compositions may be administered to any patient needing, e.g., improvement of a skin condition such as, for example, skin elasticity, skin regeneration, metabolism, etc. For example, patient populations include, but are not limited to: men over age fifty, and women over age fifty. That is, patients whose skin may not be as elastic or firm as a younger patient. However, it would be understood that humans under the age of fifty may also experience changes in their skin which would benefit from administration of a composition described herein. For example, a patient who may also be administered a composition described herein may be one who had plastic or reconstructive surgery, which population includes any human of any age. In addition to these compositions, a treating physician may add other disease-specific supplements as the patient's condition warrants. In addition to the pre-packaged nutritional supplements, the dispensing physician may add one or more other specific supplements, if needed.

Compositions comprising hyaluronic acid in combination with botulinum toxin, collagen, vitamins, minerals, biamptoprost and/or minoxidil act on the skin and/or subcutaneous muscles in order to boost cell rejuvenation and creation, alleviate fine lines and wrinkles, reduce the appearance of scars and blemishes, and improve skin clarity, elasticity, firmness, tone, vitality, and overall health.

In some embodiments, the one or more bioactive agents include, but are not limited to, B1 (thiamine), B2 (riboflavin), B3 (niacin), B5 (pantothenic acid), B6 (pyridoxine), B7 (biotin), B12 (cobalamin), collagen, botulinum toxin, platelet-rich plasma, poly-L-lactic acid, lidocaine, epinephrine and/or folic acid.

In another embodiment, the invention provides a method of making a compounded composition for the treatment of a disease or condition in a patient, comprising selecting a plurality of agents (e.g., as described herein) to compound using the system of the invention, wherein the plurality of agents are stored in a plurality of dual-function syringes.

In another embodiment, the invention provides a method for making a composition comprising compounded agents for the treatment of a disease or condition in a patient, comprising: a) storing a plurality of agent-containing dual-function syringes in an inventory structure; b) selecting one or more dual-function syringes according to the agents contained therein; c) administering a quantity of bioactive agents from the selected dual-function syringes under positive or negative pressure and transferring the agent directly to a patient or to a product container or carrier. In some embodiments, the method further comprises discarding used dual-function syringes from which the agents have been withdrawn after a single use of the dual-function syringes, wherein the dual-function syringes are limited to a single use, wherein transferring the agents to a product container comprises a means for mechanically providing a positive external pressure for moving the agent from the dual-function syringe and into the product container.

In some embodiments, the compounded agents comprise one or more of hyaluronic acid (e.g., cross-linked, non-crosslinked, or a combination thereof) neuromodulator, stem cells, an antibody, a biologic, a small molecule therapeutic, an antigen, botulinum toxin (e.g., botulinum toxin of serotype A, B, C, D, E, F or G), a vitamin, a mineral, PLLA (poly-L-lactic acid), platelet-rich plasma, retinol, retinoic acid, a bleaching/whitening agent, collagen, a neurotoxin, an anesthetic or any combination thereof.

In some embodiments, the composition comprises hyaluronic acid, botulinum toxin, and optionally one or more vitamins.

In some embodiments, the system further comprises means for administering the active agent or compounded compositions intradermally, intravenously, intramuscularly, subcutaneously, topically, orally, intranasally, by inhalation or by any other means.

A “subject” or “patient” (e.g., a mammal such as a human or a non-human animal) can be a mammal who exhibits one or more clinical manifestations and/or symptoms of a disease or condition, such as a skin condition, hair loss, wound healing and/or prevention of scarring, or for anti-aging, longevity and wellness purpose described herein. In certain situations, a subject may be asymptomatic and yet still have clinical manifestations of the disease or condition.

In one aspect, a formulation is administered until one or more symptoms are improved. In one embodiment, skin elasticity, skin regeneration, metabolism, or a combination thereof are improved by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100% following administration of one or more doses of the formulation to the patient.

In another embodiment, skin elasticity, skin regeneration, metabolism, or a combination thereof are improved by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 90-fold, about 95-fold, about 100-fold, or more, following administration of one or more doses of the formulation to the patient.

An “effective amount” is an amount sufficient to result in one or more beneficial or desired results, either partially or completely. For example, a therapeutic amount is one that achieves the desired therapeutic effect. For example, a patient may experience about a 10%, 20%, 30%, 40%, 50%, 60%, 70%, 80%, 90%, or 100% improvement in one or more symptoms. This amount can be the same or different from a prophylactically effective amount, which is an amount necessary to improve aging-related skin conditions.

An effective amount can be administered in one or more administrations, applications or dosages. A therapeutically effective amount of a therapeutic compound (i.e., an effective dosage) depends on the aesthetic and therapeutic compounds selected.

One would understand that the compositions provided herein may be administered in the described methods in a variety of dosing regimens which can be determined by the treating physician based upon the patient to be treated and the severity of the condition to be treated. Treatment of a subject with a therapeutically effective amount of the therapeutic compounds described herein can include a single treatment or a series of treatments. For example, a composition may be administered intradermally or topically to a patient on a periodic basis, for example on a daily basis, a weekly basis or as recommended depending on the judgment of the treating physician and the requirements of the individual patient or as recommended. In another example, a composition may be administered once daily for about 5 days, about 10 days, about 20 days, about 30 days, about 2 months, about 3 months, about 4 months, about 5 months, about 6 months, about 1 year, about 2 years, about 3 years, about 4 years, about 5 years or more. Alternatively, a composition may be administered once, twice, three times, about 4 times, about 5 times, about 10 times, about 20 times, about 30 times, about 40 times, about 50 times or more. Alternatively, a composition may be administered on days 1, 3, 7, 10, 14, 21, 30, 60 and 90 of a 90-day treatment period. A patient's symptoms may be monitored during treatment and the physician may alter the treatment schedule based upon one or more of the effects of the compositions.

A physician or veterinarian can readily determine and prescribe the effective amount of the formulation required. For example, the physician or veterinarian could start doses of the compounds employed in the formulation at levels lower than that required in order to achieve the desired therapeutic effect and gradually increase the dosage until the desired effect is achieved. Alternatively, a dose can remain constant.

The means by which the solution is administered to the subject, e.g., to their skin is not limiting. In some embodiments, the solution is directly applied to the skin and gently rubbed into the skin. In some embodiments, an applicator device is used to apply the solution to the skin. In some embodiments, the solution is administered to the subject's skin using a microneedle delivery device. In some embodiments, the solution is delivered to the skin with a repeated motion of the microneedle delivery device as provided herein.

In some embodiments, there is also provided a method of treating or ameliorating disease or symptoms associated with the aging process in an individual in need thereof, by administering a compounded composition comprising hyaluronic acid to the individual. In some embodiments, the composition administered further comprises one or more of cyanocobalamin or/and methylcobalamin; ascorbic acid (vitamin C); ahseutasanchin; Vitamin E; Vitamin D; selenium; zinc; glutathione (GSH); anthocyanidin; Omega-3; EPA; DHA; lecithin; CoQ10; chrome; and magnesium. In one embodiment, the composition further comprises from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to 6 mg ahseutasanchin; from about 100 to 200 IU vitamin E; from about 150 to 300 IU vitamin D; from about 100 to 150 mcg selenium; from about 30 to 50 mg glutathione (GSH); from about 100 to 150 mg anthocyanidin; from about 1 to about 500 mg omega-3; from about 200 to 300 mg EPA; from about 200 to 300 mg DHA; from about 1 to about 500 mg lecithin; from about 30 to 50 mg CoQ10; from about 100 to 150 mcg chrome; from about 150 to 200 mg magnesium.

The disease or symptom to be treated is not limiting and can include those associated with a vitamin B deficiency, such as, for example, chronic fatigue syndrome, high stress levels, pellagra, acne, microcyrtic anemia, microcytic anemia, reduced skin elasticity, impaired skin regeneration rate, slowed metabolism rate, reduced smoothness and/or softness of skin, dullness of skin, hyperpigmentation or a combination thereof.

Also provided herein is a method of improving tissue repair and regeneration in a human patient in need thereof, comprising administering to the patient an effective amount of a compositions comprising hyaluronic acid as provided herein. In some embodiments, the composition further comprises from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to 6 mg ahseutasanchin; from about 100 to 200 IU vitamin E; from about 150 to 300 IU vitamin D; from about 100 to 150 mcg selenium; from about 30 to 50 mg glutathione (GSH); from about 100 to 150 mg anthocyanidin; from about 1 to about 500 mg omega-3; from about 200 to 300 mg EPA; from about 200 to 300 mg DHA; from about 1 to about 500 mg lecithin; from about 30 to 50 mg CoQ10; from about 100 to 150 mcg chrome; and from about 150 to 200 mg magnesium. In some embodiments, the composition is administered to the patient with a microneedle delivery device.

Also provided herein is a method of improving aging-related skin conditions, such as skin elasticity, skin regeneration, metabolism, smoothness and/or softness of skin (i.e., making the skin feel smoother and softer following treatment); the overall appearance of skin; evening out skin tone and texture; clarity and/or radiance of skin; making the skin look younger; and making wrinkles appear softer and/or less prominent in human patient in need thereof, comprising administering to the patient an effective amount of a compositions comprising hyaluronic acid as provided herein. In some embodiments, the composition further comprises from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to 6 mg ahseutasanchin; from about 100 to 200 IU vitamin E; from about 150 to 300 IU vitamin D; from about 100 to 150 mcg selenium; from about 30 to 50 mg glutathione (GSH); from about 100 to 150 mg anthocyanidin; from about 1 to about 500 mg omega-3; from about 200 to 300 mg EPA; from about 200 to 300 mg DHA; from about 1 to about 500 mg lecithin; from about 30 to 50 mg CoQ10; from about 100 to 150 mcg chrome; and from about 150 to 200 mg magnesium. In some embodiments, the composition is administered to the patient with a microneedle delivery device.

The term “aging-related skin condition” relates to any skin condition or disorder associated with, caused by, or affected by, intrinsic aging and/or extrinsic aging. Aging-related skin conditions that may be treated using the present methods and formulations include, but are not limited to, wrinkles, age spots, sun damage (particularly UV radiation-induced oxidative stress), blemishes, hyperpigmented skin, age spots, increased skin thickness, loss of skin elasticity and collagen content, dry skin, lentigines, melasmas, as well as scars.

Improved skin health, includes but is not limited to improved appearance, increased regeneration, increased elasticity, increased anti-oxidative level, reduced photo-aging, reduced wrinkles, reduced scarring, reduced bacterial activities including acne, redistribution of fat and/or other content of the skin, reduced number and/or sizes of pores, reduced callusing, reduced sweating and/or body odor through skin, improved scalp health, increased hair density, increased evenness of hair growth, and increased hair strength.

In some embodiments, the compounded solution is substantially free of preservatives. In some embodiments, the solution has a viscosity that enables efficient injection with a microneedle device.

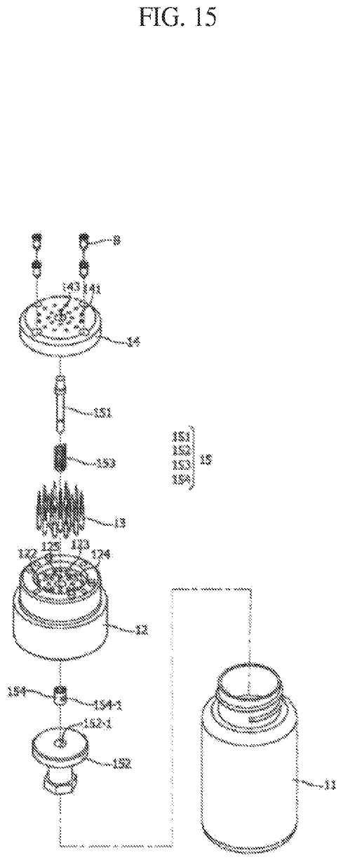

In some embodiments, the agents are administered using a microneedle delivery device. In some embodiments, the microneedle drug delivery device useful in the invention is described in U.S. Patent Appl. Pub. No. 2016/0175408, which is herein incorporated by reference in its entirety. In some embodiments, the microneedle drug delivery device is as described in Korean Patent No. 10-1582822 (see also FIG. 15 herein), which is incorporated by reference herein in its entirety.

In some embodiments, the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the bioactive composition contained in the reservoir into the skin.

In some embodiments, the means to encourage flow of the composition contained in the reservoir into the skin is selected from the group consisting of a plunger, pump and suction mechanism. In some embodiments, the means to encourage flow of the composition contained in the reservoir into the skin is a mechanical spring loaded pump system.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 2.5 mm in length and from 0.01 mm to about 0.05 mm in diameter.

In some embodiments, the microneedles are made from a substance comprising gold.

In some embodiments, the plurality of microneedles comprises an array of microneedles in the shape of a circle.

In some embodiments, the microneedles are made of 24-carat gold plated stainless steel and comprise an array of about 10 to about 50 microneedles. In some embodiments, the array comprises 20 microneedles.

In some embodiments, the microneedle delivery device is repeatedly pressed against the subject's skin to deliver the composition to the area of the skin to be treated. In some embodiments, the microneedle delivery device is repeatedly pressed about 10, about 20, about 30, about 40, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 or more times to administer the composition.

In some embodiments, the composition is administered by the microneedle delivery device with a repeated motion of penetrating the microneedle delivery device into the skin of the subject. In some embodiments, the composition is delivered into the skin by passing through the one or multiple grooves along the outer wall of the microneedle. In some embodiments, the microneedles are non-hollow.

In some embodiments, the administering comprises a repeated motion of penetrating the microneedle delivery device into the subject's skin in different areas of the subject's body.

In some embodiments, the subject's skin in the head, limbs and/or torso regions are repeatedly penetrated by the microneedle delivery device.

For example, repeated penetrations can be made in the subject's arms, legs, and torso in order to deliver the composition to different areas of the subject's body, to treat the disease or condition.

In some embodiments, the microneedle delivery device comprises a single or an array of microneedles. In some embodiments, the microneedles will have one or multiple grooves inset along its outer wall. This structural feature of the dermal delivery device allows liquids stored in a reservoir at the base of each needle to travel along the needle shaft into the tissue.

In some embodiments, the microneedle array comprises from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrlidone), etc. The microneedles will each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The microneedle delivery device is capable of delivering compositions directly to the epidermal, dermal and subcuticular layers of the skin. Therefore, it should be understood that further embodiments developed for use with non-hollow or hollow microneedle systems of delivery by those skilled in the art fall within the spirit and scope of this disclosure.

In another aspect, a microneedle device for use in the methods described herein is a device such as described in U.S. Pat. No. 8,257,324, which is hereby incorporated by reference. Briefly, the devices include a substrate to which a plurality of hollow microneedles are attached or integrated, and at least one reservoir, containing a bioactive formulation, selectably in communication with the microneedles, wherein the volume or amount of composition to be delivered can be selectively altered. The reservoir can be, for example, formed of a deformable, preferably elastic, material. The device typically includes a means, such as a plunger, for compressing the reservoir to drive the bioactive formulation from the reservoir through the microneedles, A reservoir, can be, for example, a syringe or pump connected to the substrate. A device, in some instances, comprises: a plurality of hollow microneedles (each having a base end and a tip), with at least one hollow pathway disposed at or between the base end and the tip, wherein the microneedles comprise a metal; a substrate to which the base ends of the microneedles are attached or integrated; at least one reservoir in which the material is disposed and which is in connection with the base end of at least one of the microneedles, either integrally or separably; a sealing mechanism interposed between the at least one reservoir and the substrate, wherein the sealing mechanism comprises a fracturable barrier; and a device that expels the material in the reservoir into the base end of at least one of the microneedles and into the skin. The reservoir comprises a syringe secured to the substrate, and the device that expels the material comprises a plunger connected to a top surface of the reservoir. The substrate may be adapted to removably connect to a standard or Luer-lock syringe. In one instance, the device may further include a spring engaged with the plunger. In another instance, the device may further include an attachment mechanism that secures the syringe to the device. In another instance, the device may further include a sealing mechanism that is secured to the tips of the microneedles. In another instance, the device may further include means for providing feedback to indicate that delivery of the material from the reservoir has been initiated or completed. An osmotic pump may be included to expel the material from the reservoir. One or more microneedles may be disposed at an angle other than perpendicular to the substrate. In certain instances, the at least one reservoir comprises multiple reservoirs that can be connected to or are in communication with each other. The multiple reservoirs may comprise a first reservoir and a second reservoir, wherein the first reservoir contains a solid formulation and the second reservoir contains a liquid carrier for the solid formulation. A fracturable barrier for use in the devices can be, for example, a thin foil, a polymer, a laminate film, or a biodegradable polymer. The device may further comprise, in some instances, means for providing feedback to indicate that the microneedles have penetrated the skin.

In some embodiments, the device can include, in some instances, a single or plurality of solid, screw-type microneedles, of single or varied length. Typically the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape whereby the screw threads in one embodiment lie closely together along the outer edge of the needle and, in another embodiment, the screw threads lie far from each other along the outer edge of the needle.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A device described herein may contain, in certain instances, about twenty screw thread design surgical grade microneedles. Each microneedle has a diameter that is thinner than a human hair and may be used for direct dermal application. In one instance, a microneedle has a diameter of less than about 0.18 mm. In another instance, a microneedle has a diameter of about 0.15 mm, about 0.14 mm, about 0.13 mm, about 0.12 mm, about 0.11 mm, or about 0.10 mm. Each microneedle may be plated with 24 carat gold. The device allows for targeted and uniform delivery of a composition comprising the immunizing composition into the skin in a process that is painless compared to injectables. Administration can result in easy and precise delivery of a composition comprising the immunizing composition with generally no bruising, pain, swelling and bleeding caused by the injection.

The device may include means, manual or mechanical, for compressing the reservoir, creating a vacuum, or otherwise using gravity or pressure to drive the immunizing composition from the reservoir through the microneedles or down along the sides of the microneedle. The means can include a plunger, pump or suction mechanism. In another embodiment, the reservoir further includes a means for controlling rate and precise quantity of drug delivered by utilizing a semi-permeable membrane, to regulate the rate or extent of drug which flows along the shaft of the microneedles. The microneedle device enhances transportation of drugs across or into the tissue at a useful rate. For example, the microneedle device must be capable of delivering drug at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or diameter of the bores in the microneedles, or by filling at least some of the microneedle bores with a diffusion-limiting material. It can be preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the drug composition in the reservoir. For example, in passive diffusion systems, the concentration of drug in the reservoir can be increased to increase the rate of mass transfer. In active systems, for example, the pressure applied to the reservoir can be varied, such as by varying the spring constant or number of springs or elastic bands. In either active or passive systems, the barrier material can be selected to provide a particular rate of diffusion for the drug molecules being delivered through the barrier at the needle inlet.

The array may be in any shape or combination of shapes, continuous, or discontinuous. The list of possible shapes includes, but is not limited to, circles, triangles, rectangles, squares, rhomboids, trapezoids, and any other regular or irregular polygons. The array may be attached to a reservoir to hold the substances to be delivered, and this reservoir may be any volume (e.g., about 0.25 mL to about 5 mL), shape, color, or material (glass, metal, alloy, or polymer), as determined necessary.

This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

In some embodiments, the device can include a single or plurality of solid, screw-type microneedles, of single or varied lengths housed in a plastic or polymer composite head which embodies a corrugated rubber connector. In some embodiments, the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape. The corrugated rubber connector is a unique advantage conferring feature which bestows the microneedle head with a universally adoptable feature for interfacing the micro needle cartridges with multiple glass and or plastic vials, reservoirs and containers as well as electronic appendages for an altogether enhanced adjunct liquid handling, security and surveillance utility.

In one embodiment, a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

While the above description contains many specifics, these specifics should not be considered as limitations of the invention, but merely as exemplifications of the preferred embodiments thereof. Those skilled in the art will envision many other embodiments within the scope and spirit of the invention as defined by the claims appended hereto.

What is claimed is:

1. A system for preparing active agents for personalized treatment of a subject, wherein the system comprises
i) a housing enclosing an interior space;
ii) an inventory structure comprising a plurality of chambers, wherein each chamber is capable of holding a dual-function syringe comprising a predetermined agent to be administered;
iii) means for selecting one or more dual-function syringes according to the predetermined active agent;
iv) means for moving the selected dual-function syringe to a desired location for transferring of the predetermined agent from the dual-function syringe to a product container or carrier; and
v) means for sequentially transferring a controlled quantity of the predetermined agent from each selected dual-function syringe to the product container or carrier under positive or negative pressure.

2. The system of claim 1, wherein the predetermined agent has a volume capacity of from about 0.1 to about 10.0 mL liquid.

3. The system of any of claims 1-2, wherein the predetermined agent is in a solid form in the dual-function syringe.

4. The system of any of claims 1-2, wherein the predetermined agent is in a liquid form in the dual-function syringe.

5. The system of any of claims 1-2, wherein the predetermined agent is in a semi-solid gel.

6. The system of any of claims 1-5, wherein the system further comprises and means for automatically discarding dual-function syringes from which the agents have been released.

7. The system of claim 7, wherein the syringe is discarded after a single use.

8. The system of any of claims 1-7, wherein the means for transferring the agent comprises a means for mechanically compressing the dual-function syringe to provide a positive external pressure for forcing the agent from the dual-function syringe through a needle and into the product container or carrier.

9. The system of any of claims 1-8, wherein the system is configured to limit the dual-function syringes to a single use.

10. The system of any of claims 1-9, wherein a used dual-function syringes can be disposed of within the compounding machine or manually discarded.

11. The system of any of claims 1-10, wherein the product container or carrier is selected from a receiving vessel, container, microinjection device, vial, microneedle device, syringe, and a patch for topical treatment to a subject's skin, a dropper capable of applying treatment to skin, container capable of condensing powder into an orally administered capsule or tablet, and a container capable of holding liquid which can be nasally inhaled.

12. The system of any of claims 1-11, wherein the system compounds a plurality of active agents into a single composition for administration to a subject in need of treatment.

13. The system of any of claims 1-12, wherein the system further comprises means for administering the active agent intradermally, intravenously, intramuscularly, subcutaneously, topically, orally, intranasally, or by inhalation.

14. The system of any of claims 1-13, wherein each dual-function syringe comprises
i) a syringe holder locking into place a syringe, optionally with a removable holder cap which covers the syringe;
ii) a needle rubber stopper cushioning the area between the syringe holder and the syringe;
iii) a hypodermic syringe which has a volume capacity from about 0.001 mL to about 100 mL;
iv) a plunger within the syringe;
v) optionally a removable metal needle;
vi) optionally a needle tip screwed on to the bottom of the syringe; and
vii) optionally a removable holder cap which covers the syringe.

15. The system of any of claims 1-14, further comprising a locking mechanism which attaches the syringe to the syringe holder, locking it into place.

16. The system of any of claims 1-15, wherein the system comprises 2 to about 100 chambers.

17. The system of any of claims 1-16, wherein the dual-function syringe can be attached to a needle.

18. The system of any of claims 1-16, wherein the dual-function syringe can be attached to a microneedle capable of administering liquids and/or viscous solutions.

19. The system of any of claims 1-16, wherein one or more chambers comprise a dual-function syringe comprising a powder or gel that is transferred from the dual-function syringe without a needle or needle tip to enable passage of the powder or gel through the dual-function syringe.

20. The system of any of claims 1-19, wherein the means for moving the selected dual-function syringes to a desired location comprises a manual transport system.

21. The system of any of claims 1-20, wherein the dual-function syringe comprises a circular cylindrical syringe holder, a hypodermic syringe which may or may not have a needle and needle tip screwed onto the bottom, a rubber stopper between the syringe and syringe holder, a rubber plunger within the syringe with which the agent can be transferred by providing positive external pressure, a holder cap covering the top of the syringe, and a means for pushing the plunger down.

22. The system of any of claims 1-21, wherein the syringe holder has a divert around its circumference or some other attachment mechanism which firmly locks the syringe in place as the agent is contained and administered.

23. The system of any of claims 1-22 wherein a means for discarding the used dual-function syringe comprises a waste bin positioned in the interior of the housing into which the used dual-function syringes are dropped.

24. The system of any of claims 1-24, wherein means for discarding the used dual-function syringes comprises manual transport to a waste bin.

25. The system of any of claims 1-24 wherein the needle head breaks after the first administration or extrusion rendering the dual-function syringe not reusable.

26. The system of any of claims 1-25 wherein the inventory structure comprises a carousel, wherein the chambers are arranged around a circumferential periphery of the carousel.

27. The system of claim 26, wherein the dual-function syringes are placed in the chambers, and then disposed of within the inventory structure or removed from the inventory structure.

28. The system of any of claims 1-27, wherein one or more of the chambers are pre-assigned to dual-function syringes with specific active agents.

29. The system of any of claims 1-28, wherein at least one of the chambers is capable of being personalized based on the needs of the patient.

30. The system of claim 29, wherein the personalized chamber comprises a syringe holder, capable of receiving a syringe comprising an agent suitable for the patient.

31. The system of any of claims 1-30, wherein the predetermined agents comprise one or more of bioactive agents, topical creams, nasal sprays, injectables, and pharmaceutical compounds.

32. The system of any of claims 1-31 wherein the dual-function syringes have an elongated cylindrical or prolate configuration and are horizontally or vertically oriented in respective chambers.

33. The system of any of claims 1-32, wherein the system can be used to inject an agent directly into a patient.

34. The system of any of claims 1-33, wherein means for transferring the agent comprises administering external pressure on a plunger.

35. The system of any of claims 1-34, wherein the volume of agents in the dual-function syringe that is transferred is a specific volume from about 0.001 mL to about 100 mL.

36. The system of any of claims 1-35, further comprising an injection system positioned within the housing, the injection system comprising an injection nozzle which is movable between an initial upper position and a lower position for insertion into a product container or carrier.

37. The system of claim 36, wherein the injection nozzle is also laterally movable.

38. The system of any of claims 36-37, further comprising a vacuum conduit associated with the injection nozzle.

39. The system of any of claims 1-38, wherein the means for disposing waste produced by cleaning of the dual-function syringe injection system comprises a drain leading to a waste solution container within the interior of the housing.

40. The system of any of claims 1-39, wherein means for cleaning the injection system includes a dual-function syringe containing a sterile cleaning solution comprising ethanol and/or deionized water.

41. The system of any of claims 1-40, further comprising a pressure/vacuum pump.

42. The system of any of claims 1-41, further comprising a UV sterilizing lamp positioned in the interior of the housing.

43. The system of any of claims 1-42, wherein one or more of the chambers comprises a dual-function syringe comprising one or more cannabinoid compounds.

44. The system of claim 43, wherein the cannabinoid compound comprises THC and/or CBD.

45. The system of claim 44, wherein the THC and CBD is mixed in specific ratios as prescribed for medical treatment.

46. The system of any of claims 1-45, wherein one or more of the chambers comprises a dual-function syringe comprising one or more active agents selected from hyaluronic acid (e.g., cross-linked, non-crosslinked, or a combination thereof) neuromodulator, stem cells, an antibody, a biologic, a small molecule therapeutic, an antigen, botulinum toxin (e.g., botulinum toxin of serotype A, B, C, D, E, F or G), a vitamin, a mineral, PLLA (poly-L-lactic acid), platelet-rich plasma, retinol, retinoic acid, a bleaching/whitening agent, collagen, a neurotoxin, an anesthetic or any combination thereof.

47. The system of claim 46, wherein the system makes a composition comprising a plurality of vitamins.

48. The system of claim 47, wherein the vitamins are compounded to be administered orally in solid or liquid form or by injection in liquid form.

49. The system of any of claims 1-48, wherein the chambers and dual-function syringes are color coordinated.

50. The system of any of claims 1-49, further comprising a plurality of sensors positioned in the interior of the housing for monitoring the active agent inventory and operation of the system.

51. The system of any of claims 1-50, further comprising a label printer.

52. The system of any of claims 1-51, further comprising information input and output units.

53. The system of any of claims 1-51, wherein the system is in communication with a computer.

54. The system of claim 53, wherein the computer accesses patient or health related information.

55. The system of any of claims 1-54, wherein the system is in communication with a portable electronic device.

56. The system of claim 55, wherein the computer or portable electronic device is capable of operating the system.

57. The system of claim 56, wherein the system is capable of being operated remotely by a user.

58. The system of any of claims 1-57, wherein the means for transferring the agent comprises grasping and pulling the plunger of a hypodermic syringe, and further includes means for removing the cap.

59. The system of any of claims 1-58, wherein the syringe holder and the syringe are clear glass.

60. The system of any of claims 1-59, wherein the dual-function syringes come packaged in a box containing from 1 to about 50 dual-function syringes.

61. The system of any of claims 1-60, wherein the dual-function syringes contain the same bioactive compound, different bioactive compounds, or are empty.

62. The system of any of claims 1-61, wherein the agents are administered using a microneedle delivery device, wherein the device comprises
i) a plurality of microneedles, wherein the microneedles are hollow or non-hollow, wherein one or multiple grooves are inset along an outer wall of the microneedles; and
ii) a reservoir capable of holding the solution to be delivered to a subject's skin,
wherein the reservoir is attached to or contains a means to encourage flow of the solution into the tissue; wherein the solution is capable of being delivered into the skin by passing through the one or multiple grooves along the outer wall of the microneedle by a repeated motion of penetrating the microneedle delivery device into the skin of the subject.

63. The system of claim 62, wherein the reservoir component is made of glass.

64. The system of any of claims 62-63, wherein the microneedles are non-hollow.

65. The system of any of claims 62-64, wherein the means to encourage flow of the solution into the tissue is selected from the group consisting of a plunger, pump and suction mechanism.

66. The system of any of claims 62-65, wherein the means to encourage flow of the solution into the tissue is a mechanical spring loaded pump system.

67. The system of any of claims 62-66, wherein the microneedles have a single groove inset along the outer wall of the microneedle, wherein the single groove has a screw thread shape going clockwise or counterclockwise around the microneedle.

68. The system of any of claims 62-67, wherein the microneedles are from 0.1 mm to about 1.0 mm in length and from 0.01 mm to about 0.2 mm in diameter.

69. The system of any of claims 62-68, wherein the microneedles are composed of gold.

70. The system of any of claims 62-69, wherein the plurality of microneedles comprises an array of microneedles in the shape of a circle.

71. The system of any of claims 62-70, wherein the microneedles are made of 24-carat gold plated stainless steel and comprise an array of 20 microneedles.

72. A method for compounding agents for the treatment of a disease or condition in a patient, comprising selecting a plurality of agents to compound using the system of any of claims 1-71, wherein the plurality of agents are stored in a plurality of dual-function syringes.

73. A method for making a composition comprising compounded agents for the treatment of a disease or condition in a patient, comprising: a) storing a plurality of agent-containing dual-function syringes in an inventory structure; b) selecting one or more dual-function syringes according to the agents contained therein; c) administering a quantity of bioactive agents from the selected dual-function syringes under positive or negative pressure and transferring the agent directly to a patient or to a product container or carrier.

74. The method of claim 73, further comprising discarding used dual-function syringes from which the agents have been withdrawn after a single use of the dual-function syringes, wherein the dual-function syringes are limited to a single use, wherein transferring the agents to a product container comprises a means for mechanically providing a positive external pressure for moving the agent from the dual-function syringe and into the product container.

75. The method of any of claims 72-74, wherein the compounded agents comprise one or more of hyaluronic acid (e.g., cross-linked, non-crosslinked, or a combination thereof) neuromodulator, stem cells, an antibody, a biologic, a small molecule therapeutic, an antigen, botulinum toxin (e.g., botulinum toxin of serotype A, B, C, D, E, F or G), a vitamin, a mineral, PLLA (poly-L-lactic acid), platelet-rich plasma, retinol, retinoic acid, a bleaching/whitening agent, collagen, a neurotoxin, an anesthetic or any combination thereof.

76. The method of any of claims 72-75, wherein the composition comprises hyaluronic acid, botulinum toxin, and optionally one or more vitamins.
